(19) United States
(12) Patent Application Publication
(10) Pub. No.: US 2011/0023205 A1
(43) Pub. Date: Feb. 3, 2011

(54) WELDING PROTECTION MASK

(75) Inventor: Ignaz Braendle, Mosnang (CH)

Correspondence Address: RANKIN, HILL & CLARK LLP, 38210 GLENN AVENUE, WILLOUGHBY, OH, 44094-7808, US

(73) Assignee: SPERIAN WELDING PROTECTION AG, Wattwil (CH)

(21) Appl. No.: 12/849,239

(22) Filed: Aug. 3, 2010

(30) Foreign Application Priority Data
Aug. 3, 2009 (CH) 1208/09

(51) Int. Cl. A61F 9/06

(52) U.S. Cl. 2/8.7

(57) ABSTRACT

A welding protection mask (1) includes a structural part (2) with a viewing opening (3) for the acceptance of a filter element (5) as a visor. To this end at least one insert (6) is designed at an outer surface of the structural part (2) other than the area of the viewing opening (3), which is insertable into the structural part (2) and which covers a section of the outer surface of the structural part. Thereby it is possible to individualize the welding protection masks (1) with limited effort and cost.

### BACKGROUND OF THE INVENTION

1. Field of the Invention

The invention relates to a welding protection mask as well as a locking ring according to the preamble of the corresponding independent claims.

2. Description of Related Art

A welding protection mask or a welding helmet comprises a structural part with a viewing opening, a protective shield that covers the viewing opening, and an optical filter element situated behind the protective shield. Besides its structural function, the structural part of the welding mask or welding helmet serves for the protection of the welder against mechanical impacts and against thermal radiation. This structural part usually consists of a plastic, for example of polycarbonate and is manufactured by injection molding or compression molding. The filter element is situated in the viewing opening of the structural part and serves for the optical protection of the wearer, it thus constitutes the actual visor of the device. The filter element can be passive, that is, for example it can consist of black glass. The filter element can also be active, that is, it blocks or reduces the passage of light if the intensity of the external light exceeds a predetermined threshold. The protective shield covers the viewing opening of the structural part in such a way that the filter element and, if applicable, the optical sensor are positioned behind the protective shield, that is, are positioned between the protective shield and the eyes of the welder in worn position. The protective shield is transparent and serves to protect the optical filter element from contamination and damage.

It is necessary to configure the structural part individually for a purchasing entity or for a single user. For this purpose, the structural part can be printed or furnished with stickers. As a rule however, the materials which are necessary for the protective function of the mask are difficult to print upon, above all based on the form and size of the masks. Detailed painting of the mask is expensive, stickers rapidly become unsightly and are likewise expensive.

### BRIEF DESCRIPTION OF THE INVENTION

It is therefore the object of the invention to provide a welding protection mask of the type described above which eliminates the disadvantages mentioned above.

This object is achieved by a welding protection mask that comprises inserts on an outer surface of the structural part which cover a part of the outer surface of the structural part. As the inserts are insertable, a simple individualization of the masks is possible. A defined set of masks (with respect to structural parts) of different colors, combined with a plurality of differently colored or printed inserts allows a correspondingly large number of combinations. The inserts are not only insertable, but also able to be changed out, thus the individualization can be easily altered via changing-out of inserts.

Preferably, the size of at least one insert 6 amounts to at least 10 cm² or at least 15 cm² and thereby covers a corresponding outer surface of the structural part. For example, a maximal surface amounts to 300 cm².

The insert is preferably arranged in a depression in the structural part, wherein the form of the depression corresponds to the form of the insert. Typically, the form of the depression is congruent to the form of the insert or similar (in the mathematical sense) to the form of the respective contour of the insert surface. In this way it is made less probable that any separation will occur between the structural part and the insert by objects getting between the structural part and the insert and thus detaching the insert. The depression is preferably somewhat deep, in such a way that the outer surface of the insert at least approaches being flush with the outer surface of the structural part. The surface of the structural part, which ends up lying behind the insert, remains preferably intact—with the exception of possible mounting holes. In this way, the protective function of the helmet with respect to the mask is retained even if no insert is in place. Thus, the extent to which the insert permits the passage of light is also not relevant, so that these can be manufactured from a light-colored and thus easily printable material, as a rule a plastic.

The at least one insert comprises, preferably, no structural function. It serves thus neither for a significant mechanical stabilization of the structural part, nor as a mounting point for further elements or for possible stickers or ornamentation. Typical sizes for inserts are a minimal width over 2 cm or over 4 cm, and minimal length over 5 cm, as well as maximal widths up to 10 cm or up to 15 cm as well as maximal lengths up to 10 cm or up to 20 cm or up to 30 cm.

In a preferred embodiment of the invention the inserts are of a different material than the structural part. In this way, as the inserts must not be made of the same basic material as the structural part, and the structural part takes up the optical, mechanical and thermal protective functions, (protection against impacts, resistance against hot bodies, etc.) the inserts can be manufactured from low-cost materials.

In a further preferred embodiment of the invention, the inserts comprise a different color than the structural part, and are in particular non-transparent. This can be achieved in that the material of the insert is differently colored throughout, or in that it comprises a different color visible on the outer surface. In both cases, thereby, a simple individualization is possible via choice of different colors. In the production of structural parts, thus, no individualization is required, which simplifies the production process.

For example, the structural part can be dark colored on account of its optical protection function, while the inserts can be manufactured from a light-colored plastic that is better suited to printing.

Preferably the inserts are relatively flat, and are thereby simple to print. For example, the outer surface of the inserts, when seen over the whole insert, deflects less than 3 cm, in particular less than 1 cm, from a plane.

In a preferred embodiment of the invention, the at least one insert is permanently set into the structural part. It is thus not possible to remove from the structural part without destruction thereof.

In a further preferred embodiment of the invention, the at least one insert is removably inserted into the structural part. The inserts are preferably held in place by cams, bolts or pins that are held by means of locking rings that are attached to the structural part. Thus a mechanical stability is given to the mounting, such that the inserts do not drop out, even if the mask, or respectively the helmet, is dropped. For example, the insert is exclusively held by locking rings and to this purpose are furnished four to five locking rings per insert.

In a different embodiment of the invention, one or more clips are formed on one side of the insert, which can be introduced into corresponding openings or apertures of the structural part and thereby hold the insert on this side. At the opposite side of the insert, the insert is mounted to the structural part as described, by one or more locking rings or with other holding elements. The locking rings are preferably manufactured in one piece out of plastic.

For mounting with a locking ring, each insert comprises a correspondingly formed retaining cam, which passes through an opening of the structural part and is held fast by a locking ring. The locking ring comprises a pressure section for transmitting pressure to a substantially flat surface and a seat for the accommodation of a flange of a retaining cam. The pressure section and the seat are, when viewed in the direction of an axis of the locking ring, offset relative to one another and can be compressed against one another via elastic deformation of the locking ring.

The locking ring comprises a central retaining opening, on which the seat is formed, and an insertion opening following the retaining opening, wherein the inner diameter of the retaining opening is smaller than that of the insertion opening. Thereby, a mushroom-shaped retaining cam with a flange is able to be passed through the insertion opening and snapped sideways into the retaining opening.

The described locking rings are fundamentally usable in other fields, separate from welding protection masks. They solve the problem of producing a compression in the direction of the longitudinal axis of a cam. With respect to a welding protection mask this serves for the secure holding of an insert on the thin outer shell of the structural part, in other implementations other articles with corresponding cams can be mounted to other objects.

Further preferred embodiments arise from the dependent claims.

### BRIEF DESCRIPTION OF THE FIGURES

In the following the subject of the invention will be further described with reference to preferred embodiments of the invention, which are depicted in the included drawings. Each shows schematically:

FIG. 1 three views of a welding protection mask;

FIG. 2 a detailed view of an inner side of a welding protection mask;

FIG. 3 a detailed view with cross section through snapped-in retaining cams;

FIG. 4 a locking ring with snapped-in retaining cams; and

FIG. 5 different views of and cuts through a locking ring.

The reference numerals used in the drawings and the meanings associated thereto are listed in the included reference list. As a matter of principle, in the figures, the same parts are given the same reference numerals.

### DETAILED DESCRIPTION OF THE INVENTION

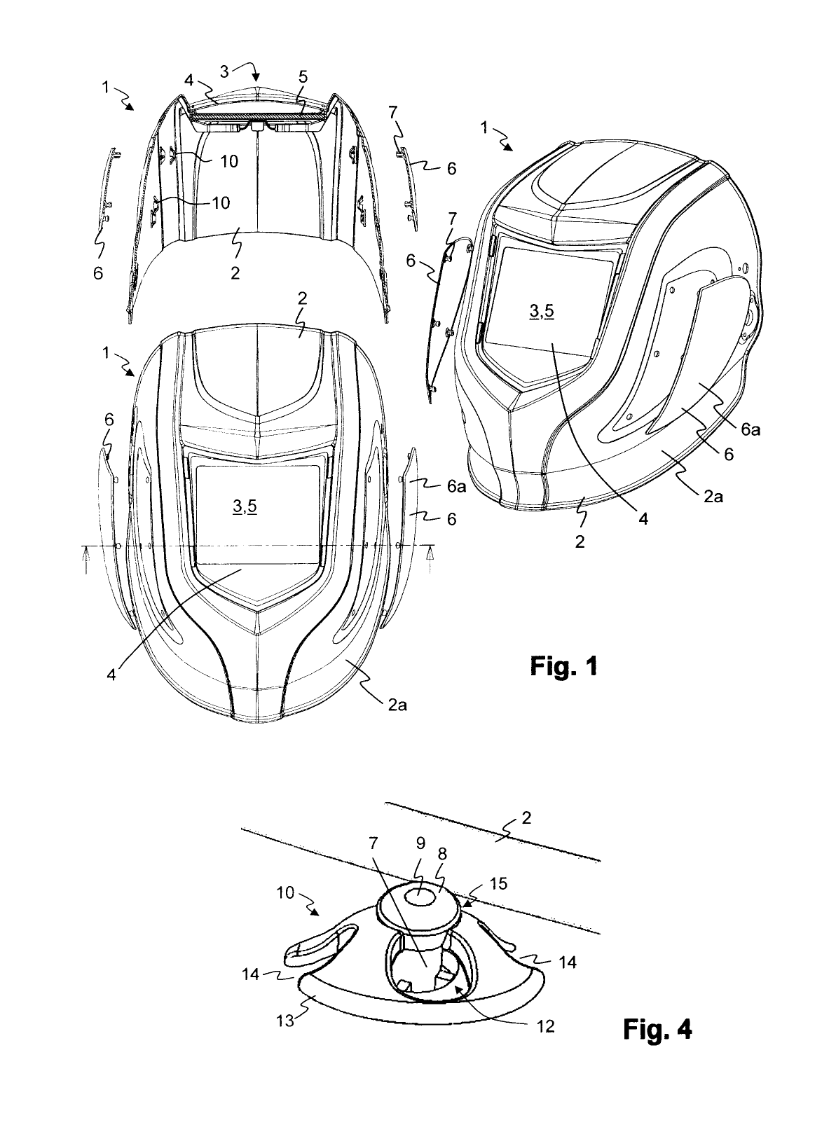

FIG. 1 depicts three views of a welding protection mask 1, in each case as exploded views: a front view (below left), a horizontal section through (above left) and a perspective view (right). A welding protection mask 1 comprises a structural part 2, also called mask body, to which the remaining elements are mounted, and which serves for protection from light, flying sparks, etc. If the upper section of the welding protection mask 1 is closed, it can also be identified as a welding helmet. On the structural part 2, in the area of a viewing opening 3, a protective shield 4 and behind this an optical filter element 5 are arranged as a visor. Furthermore, two inserts 6 are arranged as side pieces left and right of the viewing opening 3, thus in the cheek and temple area. In other embodiments of the invention only one insert is present, or yet further inserts, for example in the forehead area over the viewing opening 3, and/or in the chin area under the viewing opening 3.

The outer surface of the structural part 2 is referenced with 2a, the outer surfaces of the inserts 6 with 6a. These outer surfaces 2a, 6a preferably align at least approximately with one another, as the inserts 6 are arranged in correspondingly-formed depressions of the structural part 2, as also shown in FIG. 3.

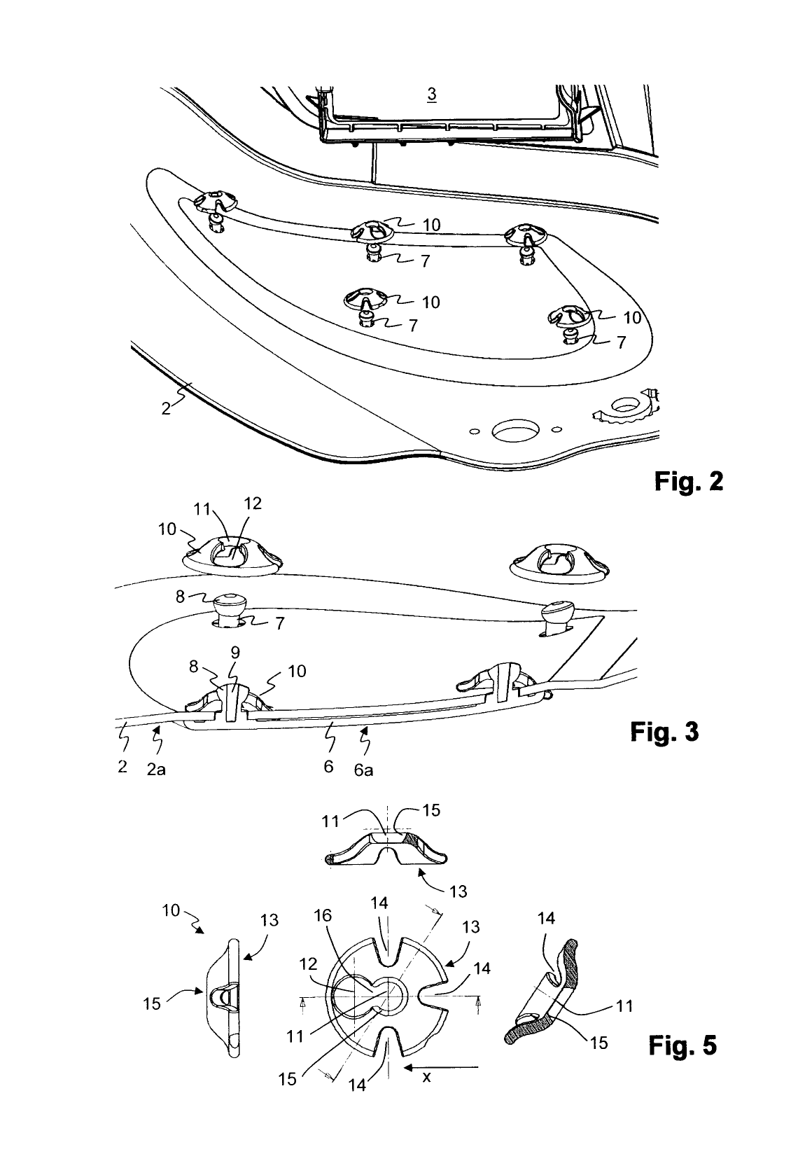

FIG. 2 depicts a detailed view of an inner side of a welding protection mask 1, wherein locking rings 10 are shown over dedicated retaining cams 7 of an insert 6 as in an exploded view. The retaining cams 7 are passed through correspondingly arranged holes of the structural part 2 and held by means of the locking rings 10. Further details pertaining to the mounting and the locking rings 10 used thereto are elucidated with respect to the following figures. FIG. 3 depicts a detailed view of a structural part 2 with an inserted insert 6, with cross sections through snapped-in retaining cams 7. FIG. 4 depicts a locking ring 10 with snapped-in retaining cams 7. The retaining cam 7 features a recess 9, in particular a central recess. FIG. 5 depicts different views of and cuts through a locking ring 10.

A locking ring 10 comprises a pressure section 13 for the application of pressure to a substantially planar or lightly curved face and a seat 15 for the holding of a, for example, mushroom-shaped flange 8 of a retaining cam 7. The pressure section 13 and the seat 15 are offset from one another with respect to the axis of the locking ring 10 and are able to be compressed against one another by elastic deformation of the locking ring 10. The locking ring 10 preferably comprises a rotationally-symmetrical basic shape, with a central axis of symmetry. The pressure section 13 is thereby spaced apart from the seat 15 in the direction of the axis of symmetry. The pressure section 13 is interrupted by cuttings or recesses 14, whereby the seat 15, under pressure upon these recesses 14, can be pressed against the pressure section 13 against the elasticity of the material of the locking ring 10.

The seat 15 is formed as an inner rim of a retaining opening 11 and its form corresponds with the form of the flange 8. The inner rim of the retaining opening 11 preferably tapers from outer to inner, similarly to the outer contour of the flange 8. The retaining opening 11 comprises on one side, beside the axis of symmetry, an opening, which merges from a connection opening 16 into an introduction opening 12 whose inner diameter is larger than the inner diameter of the retaining opening 11.

Thereby the retaining cam 7 with its flange 8 is able to be introduced through the insertion opening 12 (with a movement in the direction of the longitudinal axis of the retaining cam 7). Subsequently, the locking ring 10 is moveable through a movement normal to the longitudinal axis of the retaining cam 7 (shown in FIG. 5 by the directional arrow x). Thereby, the retaining cam 7 snaps into the retaining opening 11, as the flange 8 lies on the rim of the retaining opening 11. For the snapping-in the seat 15, under pressure on the recesses 14 is pressed against the pressure section 13, thus the locking ring 10 somewhat flattens out. The snap connection between retaining cam 7 and locking ring 10 is thus formed on the one hand from the interaction of the opening between the retaining opening 11 and the insertion opening 12 with the corresponding forms of flange 8 and seat 15, and with the elastic resilience of the seat 15 in the axial direction.

In the case of the inserted locking ring 10 the retaining opening 11 partially encloses a flange 8 of the retaining cam 7. In other words: the connection opening 16 between the insertion opening 12 and the retaining opening 11 is somewhat narrower than the thickness of the retaining cam 7 underneath the flange 8, as can be seen in the FIGS. 3 and 5. On the other hand, for snapping-in, the connection opening 16 is thus widened in order to allow the pushing-through of the retaining cam 7 in a direction perpendicular to the longitudinal axis.

The recesses 14 serve preferably not only for increasing the flexibility of the locking ring 10 in the direction of the axis of symmetry, but also for providing grip to a tool for the pushing-in and pulling-out of the locking ring 10 in a direction perpendicular to the longitudinal axis of the retaining cam 7 respectively to the axis of symmetry of the locking ring 10.

### REFERENCE LIST

1. Welding protection mask
2. Structural part
2a. Outer surface of the structural part
3. Viewing opening
4. Protective shield
5. Filter element
6. Insert
6a. Outer surface of the insert
7. Retaining cam
8. Flange
9. Concavity
10. Locking ring
11. Retaining opening
12. Insertion opening
13. Pressure section
14. Recess
15. Seat
16. Connection opening

1. A welding protection mask comprising:
a structural part with a viewing opening for the accommodation of a filter element as visor,
wherein on an outer surface of the structural part apart from the area of the viewing opening at least one insert is arranged, which is insertable in the structural part and which covers a portion of the outer surface of the structural part.

2. The welding protection mask according to claim 1, wherein the at least one insert covers at least 10 cm² or at least 15 cm² of the outer surface of the structural part.

3. The welding protection mask according to claim 1, wherein the at least one insert is arranged in a depression of the structural part, wherein the form of the depression corresponds to the form of the insert.

4. The welding protection mask according to claim 1, wherein the at least one insert comprises no structural function.

5. The welding protection mask according to claim 1, wherein the at least one insert is of a different material than the structural part.

6. The welding protection mask according to claim 1, wherein the at least one insert comprises a different color than the structural part.

7. The welding protection mask according to claim 1, wherein the at least one insert is non-transparent.

8. The welding protection mask according to claim 1, wherein the at least one insert is substantially flat, in particular the outer surface of the insert, which, when seen over the whole insert, deflects less than 3 cm from a plane.

9. The welding protection mask according to claim 1, wherein the at least one insert is irreversibly set into the structural part.

10. The welding protection mask according to claim 1, wherein the at least one insert is detachably set into the structural part.

11. The welding protection mask according to claim 10, wherein the at least one insert is mounted by at least one locking ring, by a retaining cam of the insert being introduced through an opening of the structural part and held by the locking ring.

12. The welding protection mask according to claim 11, wherein the locking ring comprises a pressure section for applying pressure to a substantially planar surface and a seat for the acceptance of a flange of a retaining cam, and the pressure section and the seat are offset from one another in the direction of an axis of the locking ring and are able to be compressed against one another by elastic deformation of the locking ring.

13. The welding protection mask according to claim 12, wherein the locking ring comprises: a central retaining opening, on which the seat is formed and an insertion opening adjacent to the retaining opening, wherein the inner diameter of the retaining opening is smaller than that of the insertion opening.

14. An insert or set of inserts which are formed for inserting into a welding protection mask according to claim 1.

15. A locking ring for the mounting of an insert to a welding protection mask according to claim 1, comprising a pressure section for the application of pressure to a substantially planar surface and a seat for the acceptance of a flange of a retaining cam wherein the pressure section and the seat are offset from one another in the direction of an axis of the locking ring and are able to be compressed against one another via elastic deformation of the locking ring.
